(19) United States
(12) Patent Application Publication
(10) Pub. No.: US 2004/0044281 A1
(43) Pub. Date: Mar. 4, 2004

(54) Systems and methods for assessing blood flow in a target tissue

(76) Inventors: John Jesberger, Highland Heights, OH (US); Jeffrey L. Duerk, Avon Lake, OH (US); Jonathan S. Lewin, Beachwood, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/441,255

(22) Filed: May 19, 2003

Related U.S. Application Data
(60) Provisional application No. 60/381,148, filed on May 17, 2002.

(51) Int. Cl. A61B 5/05

(52) U.S. Cl. 600/419

(57) ABSTRACT

A method is provided for determining an analog for blood flow in a target tissue of a subject, comprising: acquiring a baseline set of MR images prior to injection of a contrast agent into the vasculature of the subject; acquiring a set of MR images after injection of a contrast agent into the vasculature of the subject; computing two or more concentration values from the set of images; computing a derivative of the concentration values; computing the maximum value of the derivative curve to provide a value which is an analog for blood flow. An apparatus for determining an analog for blood flow in a target tissue of a subject utilizing a contrast agent, said apparatus comprising: an MRI machine for acquiring a set of MRI images prior to, during, and for a period of time following injection of the contrast agent into the vasculature of the subject; a computer component for computing an enhancement curve from the set of images; a computer component for computing a derivative curve from the enhancement curve; and a computer component for computing the maximum value of the derivative curve to provide a value which is an analog for blood flow.

### CROSS REFERENCE TO RELATED APPLICATIONS

This application claims the benefit of U.S. Provisional Patent 60/381,148 filed May 17, 2002, which is incorporated herein by reference.

### STATEMENT OF GOVERNMENT SUPPORT

This invention was supported, at least in part, by NCI grants R33CA88144 and R01CA81431, and NIH grants MO1 RR-00080 and CA83134. The United States Government may have certain rights in this invention.

### BACKGROUND OF THE INVENTION

Measuring blood flow within the body can be a useful tool in diagnosing and treating patients, especially in the field of oncology. As known to one of ordinary skill in the art, it is desirable to determine the flow of blood to a tumor or assess changes in tumor vasculature after therapy with a chemotherapeutic agent. There are a variety of known systems for obtaining blood flow information, in particular tissue perfusion. One technique for obtaining quantitative blood flow information is Positron Emission Tomography (PET). PET is not widely used due to several practical and medical disadvantages. For example, PET systems are relatively expensive to operate and require the use of a cyclotron, which is not generally available. In addition, PET requires the use of radionucleotides, which are potentially harmful to a patient. Furthermore, the anatomical resolution of PET is limited, i.e., significantly less than Magnetic Resonance Imaging (MRI). Due to this poor resolution, another imaging system is usually needed in order to assess the spatial relationship. Further, PET can only accurately determine blood flow in tumors greater than 4 centimeters.

Another technique for measuring blood flow is known as Xenon enhanced CT (computed tomography). Like PET, Xenon-enhanced CT can be uncomfortable to the patient, exposes the patient to ionizing radiation, and is limited in anatomical resolution.

Investigators have proposed that DCE-MRI might serve as a useful tool for assessing blood flow in a target tissue, particularly in a tumor. DCE-MRI is a minimally invasive technique for generating high-resolution spatial maps correlated with tissue blood flow and capillary permeability. An MRI contrast agent, typically a gadolinium-tagged macromolecule, is injected into the blood stream, where it mixes with blood plasma, crosses the capillary endothelium and diffuses into the interstitial space or extravascular extracellular fluid (EES) of the tissue. By repeated imaging, dynamic changes in the amount of contrast agent that diffuses into the EES of a target tissue can be monitored by fast MRI techniques. These changes depend on the rate of uptake of the contrast agent by the different tissues. One disadvantage to DCE-MRI is that the contrast agents approved for use are not freely diffusible, and thus uptake of the contrast agent by the different tissues is a function of blood flow F and the permeability-surface area product (PS) of the capillary endothelium with respect to the contrast agent. Further, full implementation of DCE-MRI to measure actual blood flow requires measurement of four parameters, including (i) pre-contrast longitudinal relaxivity T1₀ in arterial blood, (ii) relative contrast enhancement over time in arterial blood, (iii) pre-contrast longitudinal relaxivity T1₀ in tissue, and (iv) relative contrast enhancement over time in tissue. From these four measurements calculations of the contrast agent concentrations at each voxel can be made. Although both T1₀ and contrast enhancement over time measurements can be made in tissue, T1₀ in blood is very difficult to measure accurately, thereby diminishing the usefulness of DCE-MRI in determining blood flow in a target tissue. Accordingly, there is a need to develop new DCE-MRI based methods for assessing blood flow in a tissue.

### BRIEF DESCRIPTION OF THE DRAWINGS

The invention will be more fully understood from the following detailed description of the invention taken in conjunction with the following drawings:

FIG. 1 is a flow chart of an exemplary sequence of steps for determining the analog of blood flow in a patient.

FIG. 2 is a system diagram of the control logic for determining the analog of blood flow in a patient.

FIG. 3 is an alternate embodiment of the present invention;

FIG. 4 illustrates CT curves in which Ve, Flow F and time Tp were varied; and

FIG. 5 illustrates Gradient Peak (Gpeak), Time to gradient peak (tgp), Tgp10 is the time till the gradient drops to 10% of Gpeak and E is the enhancement concentration.

### DEFINITIONS:

ACQUISITION—the process of measuring and storing image data.

CONTRAST—the relative difference of signal intensities in two adjacent regions of an image. Image contrast is heavily dependent on the chosen imaging technique (i.e., TE, TR, TI), and is associated with such parameters as proton density and T1 or T2 relaxation times.

EXCITATION—delivering (inducing, transferring) energy into the “spinning” nuclei via radio-frequency pulse(s), which puts the nuclei into a higher energy state. By producing a net transverse magnetization an MRI system can observe a response from the excited system.

GADOLINIUM (Gd)—gadolinium is a paramagnetic contrast enhancement agent utilized in MR imaging. When injected during the scan, gadolinium will tend to change signal intensities by shortening TI in its surroundings.

DTPA—Diethylenetriaminepentaacetic acid—Gadolinium chelating (chemical bonding) agent that solves the problem of toxicity

HYDROGEN DENSITY (H+)—the concentration of Hydrogen atoms in water molecules or in some groups of fat molecules within tissue. Initial MR signal amplitudes are directly related to H+ density in the tissue being imaged.

IMAGE (DATA) ACQUISITION TIME—the time required to gather a complete set of image data. The total time for performing a scan must take into consideration the additional image reconstruction time when determining how quickly the image(s) may be viewed.

LOGIC—as used herein, includes but is not limited to hardware, firmware, software and/or combinations of each to perform a function(s) or an action(s). For example, based on a desired application or needs, logic may include a software controlled microprocessor, discrete logic such as an application specific integrated circuit (ASIC), or other programmed logic device. Logic may also be fully embodied as software.

LONGITUDINAL RELAXATION—return of longitudinal magnetization to its equilibrium value after excitation due to the exchange of energy between the nuclear spins and the lattice.

LONGITUDINAL RELAXATION TIME—the time constant, T1, which determines the rate at which excited protons return to equilibrium within the lattice. A measure of the time taken for spinning protons to re-align with the external magnetic field. The magnetization will grow after excitation from zero to a value of about 63% of its final value in a time of T1. T10 is the time constant for the recovery of longitudinal magnetization in the absence of contrast media.

MAGNETIC RESONANCE—the absorption or emission of energy by atomic nuclei in an external magnetic field after the application of RF excitation pulses using frequencies which satisfy the conditions of the Larmor equation.

MR IMAGING—the use of magnetic resonance principles in the production of diagnostic views of the human body where the resulting image is based upon three basic tissue parameters (proton density, T1 relaxation time, T2 relaxation time) and flow characteristics.

PARAMAGNETIC SUBSTANCE—a substance with weak magnetic properties due to its unpaired electrons. Researchers have developed and are developing certain paramagnetic materials, such as gadolinium, as MRI invasive contrast media.

PIXEL—acronym for a picture element, the smallest discrete two-dimensional part of a digital image display.

PROTON DENSITY—the concentration of mobile Hydrogen atoms within a sample of tissue.

PROTON DENSITY WEIGHTED IMAGE—an image produced by controlling the selection of scan parameters to minimize the effects of T1 and T2, resulting in an image dependent primarily on the density of protons in the imaging volume.

PULSE PROGRAMMER—the computer-controlled component of the MRI scanner that determines the timing of the pulse sequence parameters of the scan, such as echo time, pulse amplitude, phase and frequency.

PULSE SEQUENCE—a preselected set of defined RF and gradient pulses, usually repeated many times during a scan, wherein the time interval between pulses and the amplitude and shape of the gradient waveforms will control NMR signal reception and affect the characteristics of the MR images.

RADIO FREQUENCY—an electromagnetic wave with a frequency that is in the same general range as that used for the transmission of radio and television signals.

Abbreviated RF—The RF pulses used in MR are commonly in the 1-100 megahertz range, and their principle effect upon a body is potential tissue heating caused by absorption of the applied pulses of RF energy.

READOUT GRADIENT—magnetic field gradient applied during the period when the receiver components are on. The application of this gradient, which is active during the period when the echo is being formed, results in the frequency encoding of the object being imaged.

RECEIVER—the portion of the MRI equipment that detects and amplifies the RF signals picked up by the receiver coil. Includes a preamplifier, NMR signal amplifier, and demodulator.

RECONSTRUCTION—the mathematical process by which the displayed image is produced from the raw k-space data obtained from the receiver circuitry, typically utilizing Fourier transformation and selective filtering.

REGION OF INTEREST (ROI)—the area of anatomy being scanned that is of particular importance in the image.

RELAXATION TIME—after excitation the spins will tend to return to their equilibrium distribution in which there is no transverse magnetization and the longitudinal magnetization is at its maximum value and oriented in the direction of the static magnetic field. After excitation the transverse magnetization decays toward zero with a characteristic time constant T2, and the longitudinal magnetization returns toward equilibrium with a characteristic time constant T1.

REPETITION TIME (TR)—the amount of time that exists between successive pulse sequences applied to the same slice. It is delineated by initiating the first RF pulse of the sequence then repeating the same RF pulse at a time t. Variations in the value of TR have an important effect on the control of image contrast characteristics. Short values of TR (<1000 ms) are common in images exhibiting T1 contrast, and long values of TR (>1500 ms) are common in images exhibiting T2 contrast. TR is also a major factor in total scan time.

SLICE—the term describing the planar region or the image slice selection region.

SOFTWARE—as used herein, includes but is not limited to one or more computer readable and/or executable instructions that cause a computer or other electronic device to perform functions, actions, and/or behave in a desire manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, and/or the desires of a designer/programmer or the like.

SPATIAL RESOLUTION—the ability to define minute adjacent objects/points in an image, generally measured in line pairs per mm (1 p/mm).

TRANSVERSE RELAXATION TIME—the time constant, T2, which determines the rate at which excited protons reach equilibrium, or go out of phase with each other. A measure of the time taken for spinning protons to lose phase coherence among the nuclei spinning perpendicular to the main field due to interaction between spins, resulting in a reduction in the transverse magnetization. The transverse magnetization value will drop from maximum to a value of about 37% of its original value in a time of T2.

T2*—The effective transverse relaxation time. Faster than the spin-spin T2 decay due to external field inhomgeneities, related in the reciprocal to T2 by the relationship

$$\frac{1}{{T2}^{*}} = \frac{1}{T2} + \frac{1}{{T2}^{′}}$$

where T2′ represents the increase in the dephasing rate due to unrecoverable stochastic interactions between the excited spins and external field inhomogeneities.

VOXEL—volume element; the element of the three-dimensional space corresponding to a pixel, for a given slice thickness.

### DETAILED DESCRIPTION OF THE INVENTION

Full implementation of DCE-MRI requires measurement of both the relative contrast enhancement over time and pre-contrast longitudinal relaxivity T1₀ in both arterial blood and tissue from which calculations of gadolinium chelate concentrations at each voxel can be made. With care, both measurements can be made in tissue. However, high values of T1_{0,} like those in blood are more difficult to measure accurately, signal from blood is susceptible to several types of flow artifact, and arteries proximal to the target area are often small, sensitive to partial volume effects. Thus the acquisition of valid arterial concentration curves can be difficult. In the absence of a measured arterial input function (AIF) the use of a standardized AIF has been suggested (Tofts & Kermode, Weinman). An alternative approach is to use parameters that can be measured directly from the tissue curves, without fitting to a model which can be shown to be flow analogs. Such model-free parameters are simpler to implement, and often require shorter data collection epochs. Their desired characteristics are 1. Monotonic change with tissue blood flow that is approximately linear over the physiological range of interest. 2. Minimal variation with volume fraction Ve of the EES, and 3. If possible, minimal variation with timing of the contrast agent injection.

In accordance with the present invention, methods and systems for rapidly and easily obtaining an analog of blood flow in a target tissue are provided. The present methods and systems are especially useful for monitoring the effects of therapeutic agents that alter blood flow in tissues whose volume is subject to change as a result of administration of the therapeutic agent. One example of such tissue is tumor tissue.

Data Acquisition

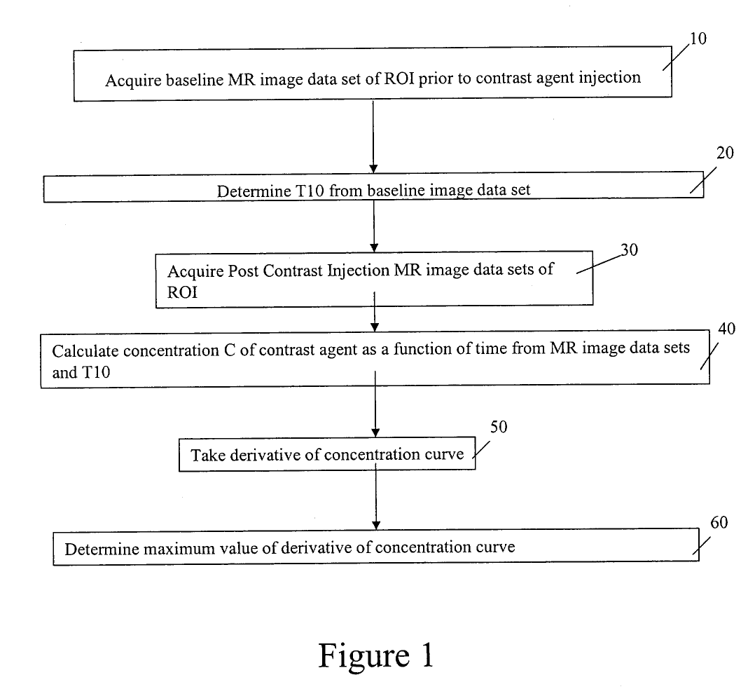

As shown in block 10 of the flow diagram of FIG. 1, the method comprises acquiring a baseline magnetic resonance (MR) image data set, which is preferably a T 1-weighted MR image data set from the region of interest prior to injection of a contrast agent into the vasculature of the subject. Acquisition of a T1-weighted image data set can be achieved by selecting a suitable TR (recovery time), Flip Angle θ and TE (echo delay time). More specifically, this is accomplished for T1-weighted images by ensuring that TE<<T2 or T2*, and that TR does not approach the upper range of T1₀ values in the target tissue. At standard clinical main field strengths of 1.5T, the nominal upper range of T1 in human soft tissue lies between 1200 and 1500 msec (Bottomley, et al, 1984). However, the fast temporal repetition rates required for DCE-MRI result in very fast TR settings of at most a few tens of milliseconds. Most soft tissue has T2 values exceeding 30 msec (Bottomley, et al, 1984). Thus for T2 sensitive sequences (e.g. Turbo Spin Echo) suitable echo times are TE<<30 msec although for T2* sensitive sequences e.g. FLASH, the constraint will be lower since T2*<T2. For the fast T1-weighted sequences employed in DCE-MRI, TE is will be set to its lowest possible value, typically on the order of a few (1-4) milliseconds. This will typically keep TE well below T2* as well. Flip angle depends on the sequence type, for example for Turbo Spin Echo FA is 90 degrees, while for gradient recalled echo sequences like FLASH, the flip angles are lower but optimal values depend on the specific TR, TE, and T1 of the target. Preferably, at least 3 T1-weighted image data sets of the region of interest are obtained prior to injection of the contrast agent into the subject. The multiple baseline images permit averaging for noise reduction in the denominator term. Also for steady state imaging techniques like FLASH, a transient component may still exist in the first acquisition and might preferably not be included in the calculations. Preferably, the region of interest is scanned at a rate of 1 slice per every 2-10 seconds.

The contrast agent is moderately to freely diffusible such that its diffusion into the EES is more dependent on flow than its permeability through the endothelium of the blood vessels in the target tissue. Preferably, the contrast agent is a paramagnetic contrast agent which enhances T1 contrast. Magnetic resonance contrast agents suitable for use in the present method are well known in the art, and are disclosed in, for example, U.S. Pat. Nos. 5,141,740; 5,078,986; 5,055,288; 5,010,191; 4,826,673; 4,822,594; and 4,770,183, which are incorporated herein by reference. Such magnetic resonance contrast agents include many different paramagnetic contrast agents, for example, gadolinium compounds. Gadopentetate dimeglumine and gadoteridol are paramagnetic gadolinium chelates that are readily available, and which rapidly redistribute into the extracellular fluid compartment. Other gadolinium compounds are acceptable, and may have a higher relaxivity, more rapid redistribution into the extracellular fluid compartment. As shown in the examples below, good results have been obtained using the contrast agent Gd-DTPA. The endothelium permeability of Gd-DTPA is fairly high, particularly in tumors, such that the rate of uptake varies predominantly with perfusion.

Preferably, Gd-DTPA is the contrast agent. The standard dosing regime for Gd-DTPA for clinical contrast enhancement imaging is 0.1 mmol Gd-DTPA per kilogram of body weight. If Gd-DTPA (Magnevist, Berlex Inc) is provided in a 0.5 molar solution for injection, the dosage is 0.2 ml of contrast agent per kilogram of body weight.

Preferably, a bolus, i.e., the entire dose, of the contrast agent is injected into the vasculature of the subject for a period of time of from about 3 seconds to about 30 seconds, more preferably for a period of time of from about 10 seconds to about 15 seconds. Preferably, the contrast agent is administered by a programmable power injector to provide strict control over the timing of the injection. Use of the programmable power injector reduces errors that could develop when the blood flow analog of the target tissue that is obtained prior to administration of a therapeutic agent, e.g. a tumor selective chemotherapeutic agent, to the subject is compared to the blood flow analog of the target tissue that is obtained after administration of the therapeutic agent to the subject.

As exemplified by Block 30 of FIG. 1, a series of MR image data sets are then obtained throughout the injection and for a sufficient period of time thereafter to obtain the blood flow analog which indicates the maximum rate of uptake of the contrast agent into the EES of the target tissue. Preferably, 2 to 10 sec temporal resolution is used for the first 90-150 sec after bolus injection of the contrast agent. The “post injection” or “post” MR image data sets as defined herein, are the MR image data sets that are acquired immediately after injection of the contrast agent is initiated. More preferably, the post MR image data sets are T1-weighted as described above, wherein each data set comprise two or more pixels, and preferably two or more voxels. Further, the baseline and post MR image data sets are preferably determined by fast imaging methods such as the FLASH method (Fast Low Angle Shot).

Data Analysis

The baseline MR image data sets that are acquired prior to injection of the contrast agent into the subject are used to determine a baseline value T₀, preferably T1_{0,} of the region of interest, as shown in Block 20 of FIG. 1. The determination of T1ₒ is well known in the art, and can be determined by conventional methods such as, but not limited to the following methods: multiple flip angle methods (e.g. Fram et al), Snapshot FLASH techniques (e.g. Haase, 1990), or variations of the Look-Locker method (Look & Locker, 1970). Once the baseline value T1ₒ is known, a tissue contrast agent concentration curve or contrast enhancement curve is determined, as shown in block 40 of FIG. 1. In one example, the tissue contrast agent concentration curve is determined from the following equations. The FLASH signal intensity for each pixel/voxel of the MR image set is physically modeled by the following equation:

$${S}_{FLASH} = {k}_{FLASH} \frac{[ sin ( θ ) \cdot {ⅇ}^{- TE / {T2}^{*}} ] \cdot [ 1 - {ⅇ}^{- TR / T1} ]}{1 - cos ( θ ) {ⅇ}^{- TR / T1}} \qquad ( 1 )$$

where kFLASH is a scaling constant that determines the range of pixel values, and the remaining expression is a scaling factor that modulates KFLASH, resulting in the actual pixel value. The repetition time TR, echo time TE, and flip angle θ are the user modifiable parameters of the FLASH pulse sequence that are used to determine the degree of T1 and T2* weighting. T2* is the dephasing rate constant that governs another mechanism of signal decay. T1 and T2* vary with the amount of contrast agent C by the following expressions:

$$\begin{matrix}\frac{1}{T1} = \frac{1}{{T1}_{0}} + {α}_{1} \cdot C & ( 2 ) \\ \frac{1}{{T2}^{*}} = \frac{1}{{T2}_{0}^{*}} + {α}_{2} \cdot C & ( 3 )\end{matrix}$$

wherein α₁ and α₂ are known constants. The right hand side of the above equations (2) and (3) were substituted into equation (1) to produce the relative signal intensity, Sᵣₑₗ:

$${S}_{rel} = \frac{{S}_{postcontrast}}{{S}_{Baseline}} = \frac{\frac{[ 1 - {ⅇ}^{- TR ( 1 / {T1}_{0} + {α}_{1} \cdot C )} ]}{[ 1 - cos ( θ ) {ⅇ}^{- TR ( 1 / {T1}_{0} + {α}_{1} \cdot C )} ]}}{\frac{[ 1 - {ⅇ}^{- TR / {T1}_{0}} ]}{[ 1 - cos ( θ ) {ⅇ}^{- TR / {T1}_{0}} ]}} \qquad ( 4 )$$

The Srel equation represents the signal intensity after the contrast agent has been injected divided by the baseline signal intensity. For strongly weighted T1 images (short TR, short TE), the variation of the exponent (−TE/T₂*) with respect to changes in the lower ranges of concentration C is negligble resulting in the cancellation of these terms from the above equation. The above equation for Srel thus represents the flash intensity after the contrast agent has been injected for a given pixel as a proportion of the baseline flash intensity (i.e., prior to the injection of contrast agent).

The above equation (4) for Srel can be solved for the concentration variable C if the baseline set of MR image data set is set equal to S_{baseline}, and the post MR sets of image data is set equal to the variable S_{postcontrast.} Concentration C can be calculated on a pixel by pixel or voxel by voxel basis and then averaged resulting in a data set of Concentration C as a function of time. A shown in Block 50 of FIG. 1, the mathematical derivative of Concentration C is determined, preferably by fitting a curve through the concentration data set and then taking the derivative of the curve equation to acquire a gradient curve. The gradient curve represents the rate of change of the tissue concentration of the contrast agent in the ROI. As shown in block 60 of FIG. 1, the maximum value of the rate of change of concentration per time (dC/dt) or Gₚₑₐₖ, is then determined. Thus, Gₚₑₐₖ, which is the magnitude of the first derivative of the concentration curve, can be employed as a flow analog that maintains a high correlation with flow with minimal sensitivity to variations in volume Vₑ. MRI acquisition techniques, coupled with image analysis techniques (e.g., intensity, rate of change of intensity), facilitate determining the efficacy of a treatment.

In one embodiment, the signal analysis and processing components of the system and method may be implemented as software executable by one or more computers or other processing devices. It may be embodied in a computer readable medium such as a magnetic disk, digital compact disk, electronic memory, persistent and/or temporary memories, and other types of memories as known in the art.

In describing the processes and methods herein, the corresponding figures and flow diagrams represent one or more exemplary methodologies of the system. As illustrated, the blocks represent functions, actions and/or events performed therein. It will be appreciated that electronic and software applications involve dynamic and flexible processes such that the illustrated blocks can be performed in other sequences different than the one shown. It will also be appreciated by one of ordinary skill in the art that elements embodied as software may be implemented using various programming approaches such as machine language, procedural, object oriented or artificial intelligence techniques. Rectangular elements in flow diagrams denote “processing blocks” and represent computer software instructions or groups of instructions. The diamond shaped elements denote “decision blocks” and represent computer software instructions or groups of instructions which affect the execution of the computer software instructions represented by the processing blocks.

Alternatively, the processing and decision blocks represent steps performed by functionally equivalent circuits such as a digital signal processor circuit or an application specific integrated circuit (ASIC). The flow diagram does not depict syntax of any particular programming language. Rather, the flow diagram illustrates the functional information one skilled in the art may use to fabricate circuits or to generate computer software to perform the processing of the system. It should be noted that many routine program elements, such as initialization of loops and variables and the use of temporary variables are not shown.

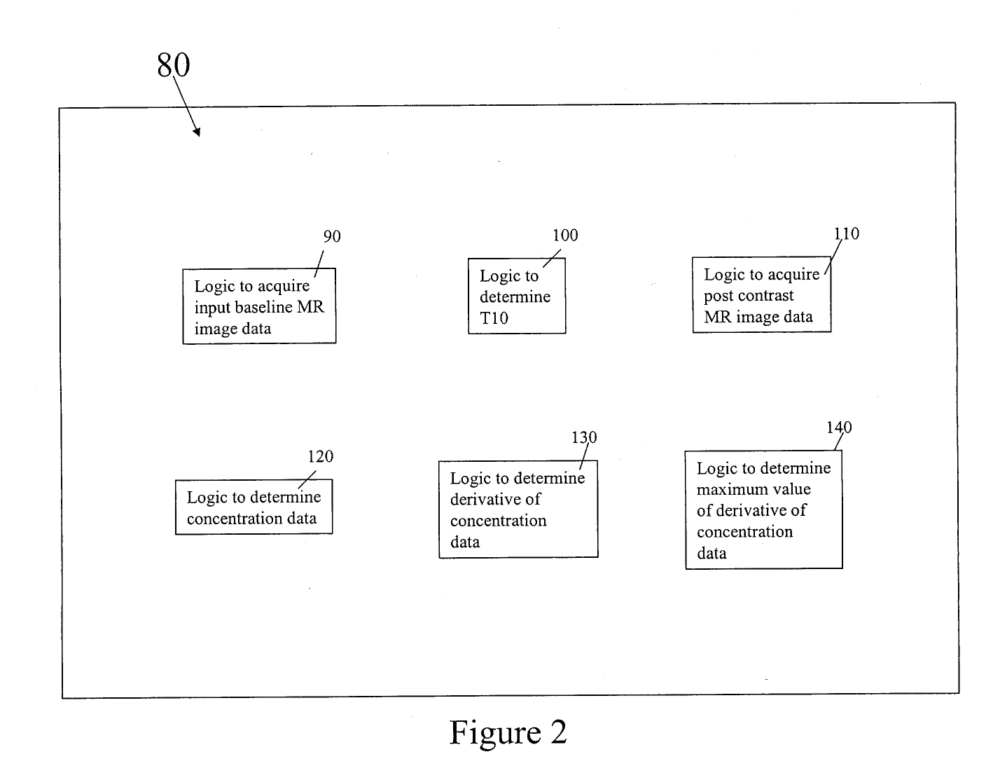

As shown in FIG. 2, the system 80 of the present invention may comprise input logic 90 for acquiring a set of MR images of the region of interest prior to the injection of a contrast agent, logic 100 for determining T10, input logic 110 for acquiring sets of MR images from the region of interest immediately following the injection of a contrast agent into the vasculature of a subject, logic 120 for determining the concentration C from the baseline MR image data sets, the post injection MR image data sets and T10. The system 80 further comprises logic 130 to determine the derivative of the concentration data set and logic 140 to determine the maximum value of the derivative of the concentration data set to provide a value which is an analog of blood flow in the region of interest.

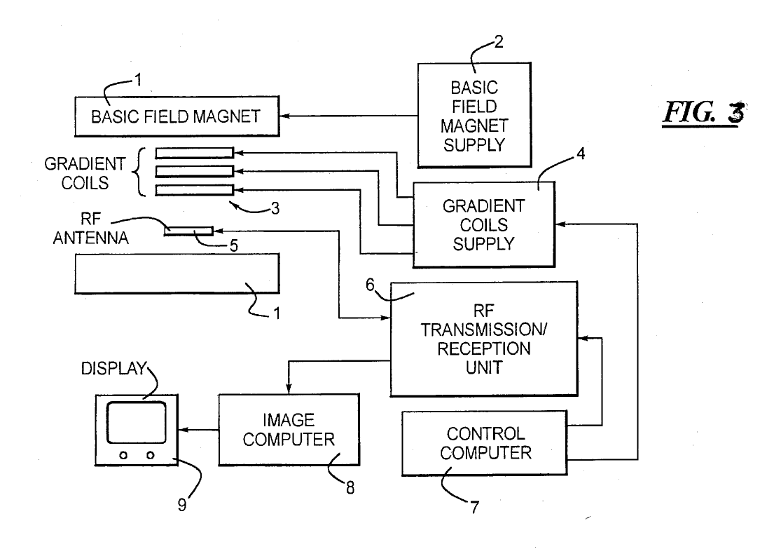

The system 80 may optionally comprise a magnetic resonance apparatus as shown in FIG. 3. The apparatus includes a basic field magnet 1 and by a basic field magnet supply 2. The system has gradient coils 3 for respectively emitting the gradient magnetic fields G_{S}, G_{P} and G_{R}, operated by a gradient coil supply 4. A radio frequency (RF) antenna 5 is provided for generating the RF pulses, and for receiving the resulting magnetic resonance signals from an object being imaged. The RF antenna 5 is operated by an RF transmission/reception unit 6. The gradient coil supply and the RF transmission/reception unit 6 are operated by a control computer 7 to produce radio frequency pulses which are directed to the object to be imaged. The magnetic resonance signals received from the RF antenna are subject to a transformation process, such as a two dimensional fast Fourier Transform, which generates pixelated image data. The transformation can be performed by an image computer 8 or other similar processing device. The image data may then be shown on a display 9.

### EXAMPLE 1

A series of Kety model simulations were performed to examine five candidate model-free parameters, some taken from previously published studies, in light of the following desired characteristics: 1. Monotonic change with tissue blood flow that is approximately linear over the physiological range of interest. 2. Minimal variation with volume fraction Ve of the EES, and 3. If possible, minimal variation with timing of the contrast agent injection.

We then examined these parameters in a cohort of patients from a phase I trial of the novel anti-tumor vascular targeting agent disodium combretastatin-A-4-3-O-phosphate [combretastatin A-4 phosphate (CA4P)].

Methods

Kety Simulation. The tissue contrast curves C_{T}(t) were generated using the modified Kety equation (3).

$$\frac{ⅆ {C}_{T}}{ⅆ t} = E \cdot F \cdot ρ [ {C}_{A} - ( 1 - Hct ) \frac{{C}_{T}}{{V}_{e}} ]$$

F is flow, E is the extraction fraction of the contrast agent from the Renkin-Crone equation,

$$E = 1 - {ⅇ}^{- \frac{F}{PS}}$$

ρ is tissue density (approximated as 1 g/cc for simplicity), Ve is the volume fraction of the EES, and Hct=0.44 was the assumed hematocrit.

The AIF was modeled by

$${C}_{A} ( t ) = A \cdot ( \frac{t}{{t}_{p}^{2}} ) \cdot exp ( - \frac{t}{{t}_{p}^{2}} ) + B \cdot ( 1 - exp ( \frac{t}{{t}_{p}^{2}} ) )$$

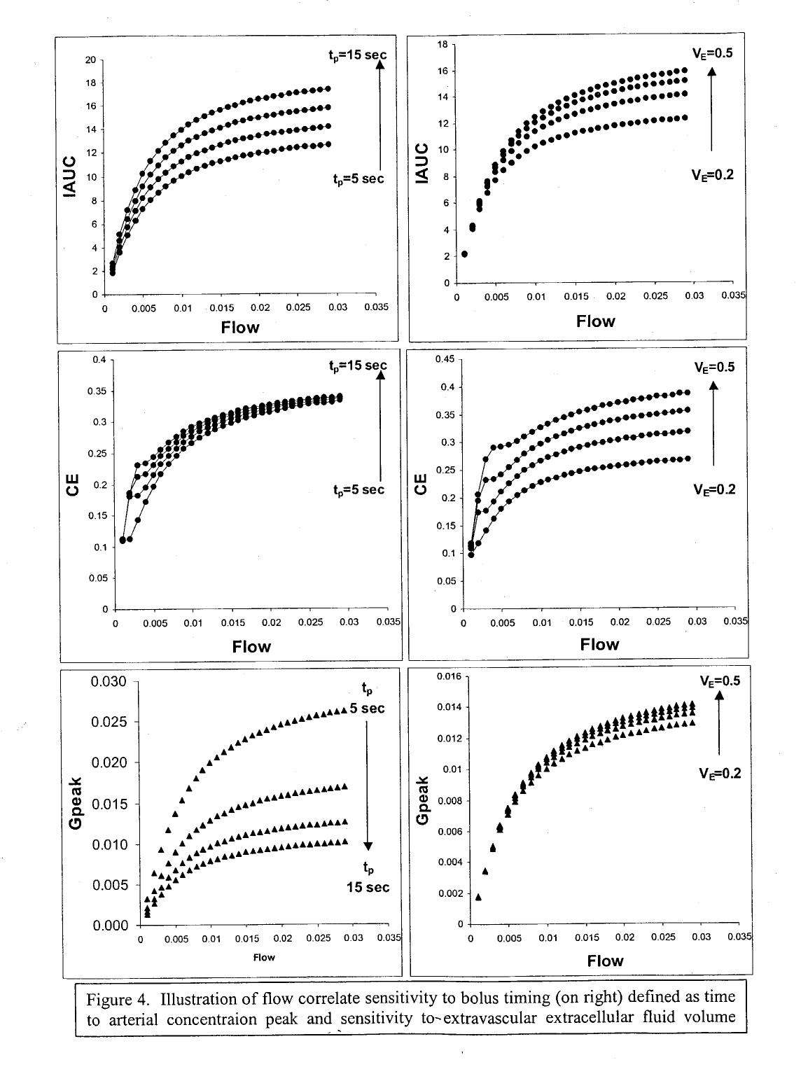

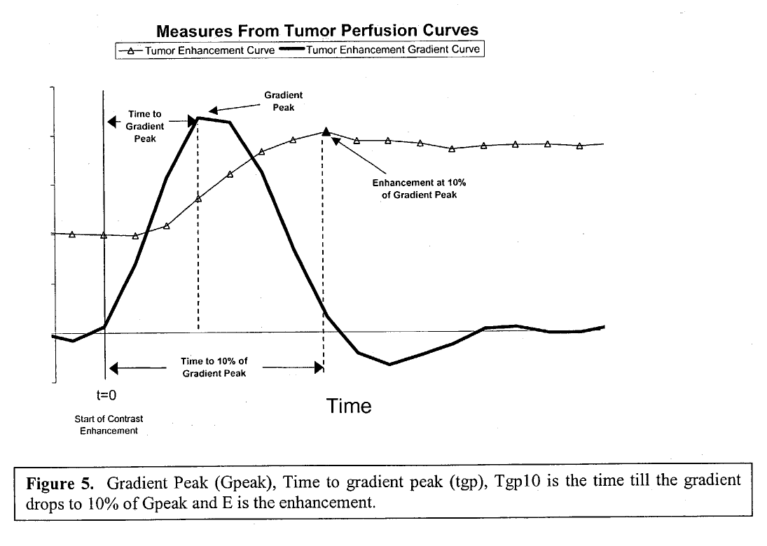

With A=40 mMol·sec (the area under curve without recirculation), B=0.5 mMol (the equilibrium concentration), and tp was the time to peak. As shown in FIG. 4, CT curves were simulated using the Runge-Kutta technique, first holding tₚ=10 sec, while varying Vₑ (0.2 to 0.5), then holding Vₑ=0.35 while varying tₚ (5 to 15 seconds). In each case flow F was varied from 0.001 to 0.03 ml/sec/g. Five parameters were calculated from each curve as shown for example purposes in FIG. 5. Magnitude of the first derivative peak (Gₚₑₐₖ), time from bolus arrival to first derivative peak (t_{gp,}) time for gradient to drop to 10% of peak value (t_{gp10,}), the concentration at t_{gp10} (denoted CE), and initial area under the C_{T} curve from bolus arrival to 60 sec afterwards (IAUC) (Evelhoch, 1999.

DCE-MRI. Six patients were studied using a 1.5T scanner (Siemens Magnetom Vision). Perfusion studies were obtained with a single-slice Fast Low Angle Shot (FLASH) sequence (TR=10, TE=4, FA=30°, slice thickness=10 mm, matrix=128×256, FOV=16×25 cm-35×35 cm) repeated 128 times, once every 2.6 seconds. The first 10 images were obtained without contrast enhancement, after which an approximately 10 second bolus of Gadolinium-DTPA contrast agent (Magnevist, Berlex Laboratories) was administered intravenously. For this sequence the T2 shortening effects of this agent were shown to be negligible across tissue concentrations obtainable at this dose. DCE-MRI was performed twice on each patient; once to obtain a pre-treatment baseline, and once 4-6 hrs after infusion of combretestatin. The slice position and orientation between days within each patient was carefully matched to obtain data from the same region of interest (ROI) in the tumor. Image processing was performed off-line using a custom software package that permitted a trained rater to view all 128 images in a study and to interactively identify the tumor ROI.

Results Simulation. Gₚₑₐₖ, CE, and IAUC from each simulation were plotted against F for different fixed values of tₚ and Vₑ (figure). Each increased monotonically with F with minimal nonlinearity for F>0.01 ml/sec/g. IAUC and Gₚₑₐₖ were the most sensitive to changes in tₚ, while CE was least affected. Conversely, CE was extremely sensitive to Vₑ followed by IAUC, while Gₚₑₐₖ was minimally sensitive. The variation of t_{gp10} vs. flow was highly nonlinear, while the variation of t_{gp} with flow was too small to make it a good analog (plots of t_{gp} and t_{gp10} vs. flow are not shown).

DCE-MRI. A summary table of all variables, pre and post treatment, and the change observed in each for each patient is shown in the Table. Time to gradient peak t_{gp} was not statistically significantly different between pre- and post-treatment, indirectly supporting the conclusion that there were no systematic differences in contrast agent administration (Wilcoxon Z=0.730, p=0.47). Gradient peak (Gₚₑₐₖ), the flow dependent parameter least sensitive to variations in EES volume fraction, was statistically significantly reduced after treatment (Wilcoxon Z=2.20, p=0.028), as was enhancement E, (Z=-2.20, p=0.028). This was expected since both parameters increased monotonically with blood flow in simulations in the two compartment model simulations. The last parameter, t_{gp10} was not statistically different between pre- and post-treatment scans, although this could well be due to the nonlinear relationship with flow, and its sensitivity to possible changes in both the EES volume fraction and the arterial input function.

Discussion and Conclusions: We conclude that Gₚₑₐₖ is a flow analog that maintains high correlation with flow with minimal sensitivity to variation in Vₑ. We believe that Gₚₑₐₖ should be strongly considered as an index of flow in investigations where variations in the EES volume fraction Vₑ might occur, such as in the presence of inflammation, apoptosis, or tumor growth, and particularly when comparing across long intervals between tissue measurements. In addition, our results confirm, as others have shown, that flow correlates are highly sensitive to the timing of the contrast administration. Thus, it is highly desirable that the timing of contrast administration be strictly controlled.

### REFERENCES

1. Folkman J. Tumor angiogenesis: therapeutic implications. N Engl J Med 285:1182-6, 1971.

2. Folkman J. Clinical applications of research on angiogenesis. N Engl J Med 333:1757-63, 1995.

3. Denekamp J and Hill S A. Angiogenic attack as a therapeutic strategy for cancer. Radiother Oncol 20 (suppl): 103-12, 1991.

4. Tofts, et al. (1997) JMRI 7:91-101.

5. Evelhoch, (1999) JMRI, 10:254-259

6. Tofts, PS et al (1999): JMRI, 10:223-232.

7. Renkin (1959) Am J Physiol, 197:1205-1210

8. Fram E K, Herfkens R J, Johnson G A, Glover G H, Karis J P, Shimakawa A, Perkins T G, Pelc N J. Rapid calculation of T1 using variable flip angle gradient refocused imaging. Magn Reson Imaging 1987;5(3):201-8.

9. Look, D C, Locker, D R. Time saving in measurement of NMR and EPR relaxation times. Rev Sci. Instrumen. 41:250-251, 1970.

10. Haase, A. Snapshot FLASH MRI: Applications to T1, T2, and chemical shift imaging. Magn. Reson. Med. 13:77-89,1990.

11. Bottomley P A, Foster T H, Argersinger R E, Pfeifer L M. A review of normal tissue hydrogen NMR relaxation times and relaxation mechanisms from 1-100 MHz: dependence on tissue type, NMR frequency, temperature, species, excision, and age. Med Phys 1984 July-August;11(4):425-48

TABLE I
Summary of pre- and post-treatment MR tumor blood flow estimation in 7 patients treated with
combretastatin (CA4P). Bold italics were used when the direction of change was that expected
in association with decreased vascular perfusion.

| Patient | Dose | Pre-treatment | | | | Post-treatment | | | | Change | | | |
|---|---|---|---|---|---|---|---|---|---|---|---|---|---|
| Number | (mg/m²) | t_{gp} | Gₚₑₐₖ | t_{gp10} | E | t_{gp} | Gₚₑₐₖ | t_{gp10} | E | t_{gp} | Gₚₑₐₖ | t_{gp10} | E |
| 16 | 60 (1 hr) | 5.24 | 3.63 | 36.60 | 1.50 | 7.86 | 2.19 | 20.90 | 1.28 | 2.62 | −1.44 | −15.70 | −0.22 |
| 17 | 60 (1 hr) | 15.70 | 3.23 | 31.40 | 1.50 | 11.70 | 1.48 | 52.60 | 1.30 | −4.00 | −1.75 | 21.20 | −0.20 |
| 19 | 60 (1 hr) | 7.86 | 8.69 | 18.30 | 1.99 | 7.86 | 4.93 | 36.60 | 1.72 | 0.00 | −3.76 | 18.30 | −0.27 |
| 21 | 60 (1 hr) | 13.10 | 4.62 | 39.30 | 1.82 | 15.70 | 3.57 | 44.50 | 1.76 | 2.60 | −1.05 | 5.20 | −0.06 |
| 22 | 60 (1 hr) | 7.86 | 5.00 | 34.00 | 1.93 | 13.10 | 3.47 | 44.50 | 1.80 | 5.24 | −1.53 | 10.50 | −0.13 |
| 23 | 60 (10 min) | 11.50 | 7.30 | 39.10 | 2.42 | 11.50 | 6.18 | 25.30 | 2.01 | 0.00 | −1.12 | −13.80 | −0.41 |
| 24 | 60 (10 min) | 6.90 | 4.79 | 39.10 | 1.82 | 6.90 | 5.71 | 34.50 | 1.87 | 0.00 | 0.92 | −4.60 | 0.05 |

1. A method for determining an analog for blood flow in a target tissue of a subject, comprising:
acquiring a baseline set of MR images prior to injection of a contrast agent into the vasculature of the subject;
acquiring a set of MR images after injection of a contrast agent into the vasculature of the subject;
computing two or more concentration values from the set of images;
computing a derivative of the concentration values;
computing the maximum value of the derivative curve to provide a value which is an analog for blood flow.

2. The method of claim 1 wherein the contrast agent is Gd-DTPA.

3. The method of claim 1 wherein the contrast agent is injected into the vasculature for a period of time about equal to or less than 30 seconds.

4. The method of claim 1 wherein the images are T1 weighted.

5. A method for determining an analog of blood flow in a region of interest in a target tissue, comprising:
(a) acquiring a series of images from the region of interest in the target tissue prior to, and for a period of time following injection of a contrast agent into the vasculature of the subject;
(b) processing the series of images to provide a tissue enhancement curve for the region of interest; and
(c) calculating the Gₚₑₐₖ of the derivative of the tissue enhancement curve to provide an analog of blood flow in the region of interest.

6. A method of determining the efficacy of a therapeutic agent in a target tissue in a subject, wherein said therapeutic agent is a tumor vasculature chemotherapeutic agent, an anti-angiogenic agent, or an anti-ischemic agent, the method comprising:
a) determining a first analog of blood flow in a region of interest in the target tissue using the method of claim 2;
b) administering the therapeutic agent to the subject;
c) determining a second analog of blood flow in said region of interest in said target tissue using the method of claim 2;
d) comparing the first analog of blood flow to the second analog of blood flow, wherein an alteration in the value of the second analog of blood flow as compared to the first analog of blood flow indicates an effect of the therapeutic agent on blood in the region of interest in the target tissue.

7. A method of determining the efficacy of a therapeutic agent in a target tissue in a subject, wherein said therapeutic agent is a tumor vasculature chemotherapeutic agent, an anti-angiogenic agent, or an anti-ischemic agent, the method comprising:
(a) acquiring a series of images from the region of interest in the target tissue following injection of a contrast agent into the vasculature of the subject;
(b) processing the series of images to provide a tissue concentration values for the region of interest; and
(c) determining the efficacy of the treatment based, at least in part, on at least one of,
the concentration values, the derivative of the concentration values, and the maximum value of the derivative values.

8. An apparatus for determining an analog for blood flow in a target tissue of a subject utilizing a contrast agent, said apparatus comprising:
an MRI machine for acquiring a set of MRI images prior to and for a period of time following injection of the contrast agent into the vasculature of the subject;
logic for computing one or more concentration values from the set of images;
logic for computing derivative values from the concentration values;
and logic for computing the maximum value of the derivative values to provide an analog for blood flow.

9. A computer readable medium for storing computer executable instructions operable to perform computer executable elements of the method of claim 1.

10. A system for determining an analog for blood flow in a target tissue of a subject, comprising:
input logic for acquiring a baseline set of MR images prior to injection of a contrast agent into the vasculature of the subject;
input logic for acquiring a set of MR images after injection of a contrast agent into the vasculature of the subject;
logic for computing two or more concentration values from the set of images;
logic for computing a derivative of the concentration; and
logic for computing the maximum value of the derivative curve to output a value which is an analog for blood flow.
